(19) United States
(12) Patent Application Publication
(10) Pub. No.: US 2013/0310738 A1
(43) Pub. Date: Nov. 21, 2013

(54) PORTABLE INTRAVENOUS FLUID DELIVERY DEVICE WITH A USER INTERFACE

(71) Applicant: Anup Dev T. SALGIA, Akron, OH (US)

(72) Inventor: Anup Dev T. SALGIA, Akron, OH (US)

(73) Assignee: LIFEMEDIX, LLC, Akron, OH (US)

(21) Appl. No.: 13/476,411

(22) Filed: May 21, 2012

(51) Int. Cl. A61M 5/168; A61M 5/142

(52) U.S. Cl. 604/67; 604/151

(57) ABSTRACT

A fluid delivery device includes a first housing and a second housing removably connected to the first housing. The fluid delivery device further includes an input line operatively connected to a fluid source that is external to each of the first housing and the second housing. A fluid reservoir is operably connected to the input line, and an output line is operably connected to the fluid reservoir. A pump is configured to facilitate a flow of fluid from the input line to the fluid reservoir and from the fluid reservoir to the output line. The fluid delivery device further includes a motor operably connected to the pump, a user interface, and a power source operably connected to the motor and to the user interface.

### FIELD OF INVENTION

The present application relates to devices for infusing intravenous fluids. More particularly, the present application relates to a portable device for infusing intravenous fluids into a subject.

### BACKGROUND

In the medical and veterinary setting, the need may arise to rapidly infuse intravenous fluid into a subject. Saline and lactated ringer's solution are examples of commonly used intravenous fluids. Such fluids may be used to maintain or elevate blood pressure and promote adequate perfusion. In the shock-trauma setting or in septic shock, fluid resuscitation is often first-line therapy to maintain or improve blood pressure.

Currently, first responders, such as emergency medical technicians or military field medics, are known to administer intravenous fluids with a gravity drip, having a fluid bag, a fluid line, and a needle or intravenous catheter. When the needle or intravenous catheter is inserted into a subject, gravity causes the fluid to flow from the fluid bag, through the fluid line and needle, and into the subject. To increase the speed at which intravenous fluids are infused into the subject, the technician may apply pressure on the bag. Pressure may be applied by hand, by employing a blood pressure cuff, or other external pneumatic pressure device on the fluid bag itself

Additionally, intraosseous (I.O.) lines have gained wider use in pediatric subjects, as well as adult subjects. Intraosseous infusion is a process of injection directly into the marrow of a subject's bone. Intraosseous lines often have a relatively slow rate of infusion.

### SUMMARY OF THE INVENTION

In one embodiment, a portable intravenous fluid delivery system includes an actuator housing and a reservoir housing removably attached to the actuator housing. The actuator housing includes a motor, a controller operably connected to the motor, and a user interface operably connected to the controller. The controller is configured to actuate the motor and to control a speed of the motor. The reservoir housing includes at least one fluid input line, having a first end and a second end. The first end is configured to be operably connected to a fluid source that is external to the actuator housing and external to the reservoir housing. The reservoir housing further includes at least one fluid reservoir operably connected to the second end of the at least one fluid input line, and at least one fluid output line operably connected to the at least one fluid reservoir. At least one pump is configured to facilitate a first flow of fluid from the at least one fluid input line to the at least one fluid reservoir and a second flow of fluid from the at least one fluid reservoir to the at least one fluid output line. The at least one pump is operably connected to the motor. The reservoir housing also includes a power source operably connected to the motor in the actuator housing.

In another embodiment, a fluid delivery device includes a first housing and a second housing removably connected to the first housing. The fluid delivery device further includes an input line operatively connected to a fluid source that is external to each of the first housing and the second housing. A fluid reservoir is operably connected to the input line, and an output line is operably connected to the fluid reservoir. A pump is configured to facilitate a flow of fluid from the input line to the fluid reservoir and from the fluid reservoir to the output line. The fluid delivery device further includes a motor operably connected to the pump, a user interface, and a power source operably connected to the motor and to the user interface.

In yet another embodiment, a portable intravenous fluid delivery kit includes a first housing, a second housing, a fluid bag external to the first housing and second housing, and a fluid line having a first end connected to the fluid bag and a second end connected to the second housing. The first housing includes an electric motor and a user interface. The second housing includes a fluid input, a fluid reservoir operably connected to the fluid input, a fluid output operably connected to the fluid reservoir, a pump configured to be connected to the electric motor, and a power source configured to be connected to the electric motor and the user interface.

### BRIEF DESCRIPTION OF THE DRAWINGS

In the accompanying drawings, structures are illustrated that, together with the detailed description provided below, describe exemplary embodiments of the claimed invention.

In the drawings and description that follows, like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 is a schematic drawing of an intravenous fluid delivery device in combination with a fluid bag and a fluid line;

FIG. 2 is an exploded perspective view of one embodiment of an intravenous fluid delivery device having an actuator housing and a reservoir housing;

FIGS. 3A and 3B are cross-sections of one embodiment of a reservoir housing having a pair of asymmetric diaphragm pumps;

FIGS. 4A and 4B are cross-sections of one embodiment of a reservoir housing having a pair of symmetric diaphragm pumps;

FIG. 5 is a cut away view of an alternative embodiment of a pump for use in an intravenous fluid delivery device;

FIG. 6 is a cross section of a fluid line housing for an intravenous fluid delivery device;

FIG. 7 is an alternative embodiment of a fluid line housing for an intravenous fluid delivery device;

FIG. 8 is a front perspective view of one embodiment of an intravenous fluid delivery device having a user interface;

FIG. 9 is an exploded rear perspective view of the intravenous fluid delivery device of FIG. 8;

FIG. 10 is front perspective view of an alternative embodiment of an intravenous fluid delivery device having a user interface;

FIG. 11 is an exploded rear perspective view of the alternative intravenous fluid delivery device of FIG. 10;

FIG. 12 is front perspective view of another alternative embodiment of an intravenous fluid delivery device having a user interface;

FIG. 13 is an exploded rear perspective view of the alternative intravenous fluid delivery device of FIG. 12; and

FIGS. 14A-F are schematic drawings of exemplary displays of a user interface.

### DETAILED DESCRIPTION

Multiple embodiment of intravenous fluid delivery devices are shown and described herein. It should be understood that the disclosed fluid delivery devices may be employed to deliver any known intravenous fluids, including, without limitation, saline, lactated ringer's solution, colloid solution, platelets, and blood. Further, the use of the disclosed fluid delivery devices is not limited to the intravenous application of fluids. It should be understood that the fluid delivery devices may be used, for example, for wound irrigation or other cleaning or sterilization purposes. For such uses, the fluid delivery devices may be used with water, alcohol, or other sterilants.

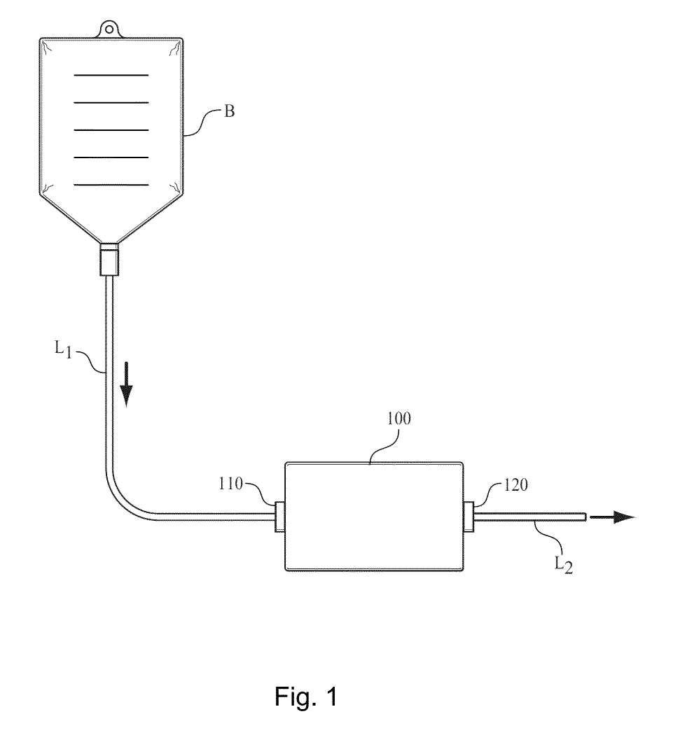

FIG. 1 is a schematic drawing of an intravenous fluid delivery device 100 in combination with a fluid bag B and a fluid line L. In the illustrated embodiment, the fluid line L includes a first line L₁ and a second line L₂. The first fluid line L₁ is connected to an output of the fluid bag B and an input 110 of the intravenous fluid delivery device 100. The first fluid line L leads to an internal fluid reservoir (not shown) in the intravenous fluid delivery device 100. The intravenous fluid delivery device 100 further includes one or more mechanisms (not shown) to facilitate the flow of fluid through the internal fluid reservoir. In one embodiment, the input 110 of the intravenous fluid delivery device 100 is a one-way valve. In alternative embodiments, the input may be a 2-way valve, or an adjustable, bi-directional valve.

The second fluid line L₂ is connected to an output 120 of the intravenous fluid delivery device 100 and leads to a subject, usually by a needle or intravenous catheter. Alternatively, the intravenous fluid delivery device 100 may employ central line catheters and interosseous lines. In one embodiment, the output 120 is also a one-way valve. One-way valves allow the fluid only to flow from the fluid bag B, to the subject, and not in a reverse direction. In alternative embodiments, however, the output may be a 2-way valve, or an adjustable, bi-directional valve.

The intravenous fluid delivery device 100 may be used in-line (i.e., in series) as described above. Alternatively, the intravenous fluid delivery device 100 may also be used in a bypass-type configuration (i.e., in parallel) to allow a gravity drip to continue.

The intravenous fluid delivery device 100 further includes a facilitating component (not shown), configured to force fluid from the input 110 of the intravenous fluid delivery device 100 to the output 120.

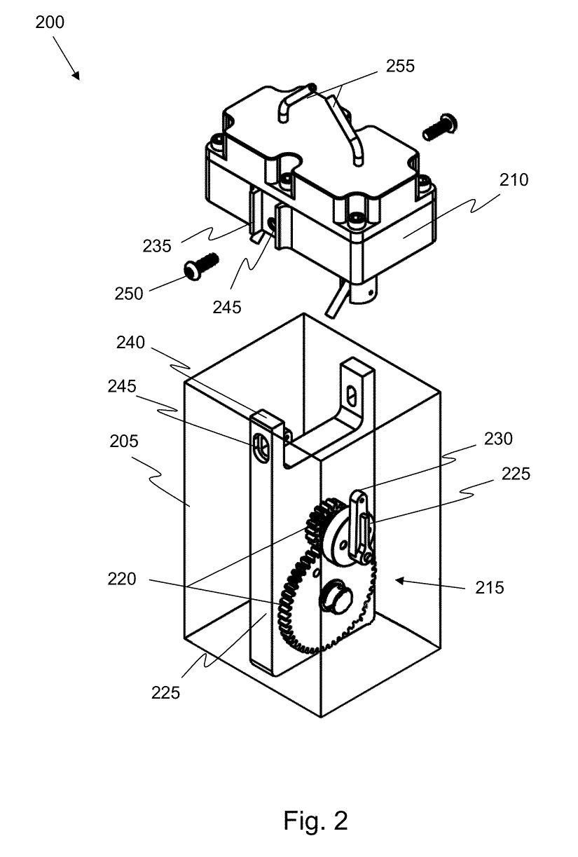

FIG. 2 illustrates an exploded perspective view of an intravenous fluid delivery device 200 having an actuator housing 205 and at least one reservoir housing 210. The actuator housing 205 includes an electric motor 215 having a series of gears 220 mounted on a base 225. While two gears are shown in the illustrated embodiment, it should be understood that three or more gears may be employed. Alternatively, gears may be omitted.

The series of gears 220 rotates a disc 225 having a plunger 230 pivotally attached thereto. The plunger is one example of a facilitating member configured to facilitate a flow of fluid through a reservoir. The plunger 230 is configured to operatively connect to a pump (not shown) in the reservoir housing 210. The actuator housing 205 may further include a second disc and plunger (not shown) mounted on the opposite side of the base 225 and configured to operatively connect to a second pump (not shown) in the reservoir housing 210.

In the illustrated embodiment, the gears 220 have a fixed gear ratio. In an alternative embodiment (not shown), a gear shift mechanism may be employed to vary the gear ratio. In such an embodiment, an operator may choose to shift gears to increase or decrease the flow of fluid.

The reservoir housing 210 may be configured to be removably attached to the actuator housing 205. In such an embodiment, the reservoir housing 210 may be removed and replaced with a replacement reservoir housing (not shown). For example, the reservoir housing 210 may be replaced after each use for sterility or safety reasons, or to comply with FDA standards, hospital standards, or other standards. In such an embodiment, the reservoir housing 210 may be kept in sterile packaging prior to use. Additionally, the reservoir housing 210 may be filled with fluid prior to packaging, such that no priming is required when a new reservoir housing 210 is attached to the actuator housing 205. In an alternative embodiment (not shown), the reservoir housing may be permanently attached to the actuator housing.

In the illustrated embodiment, the reservoir housing 210 includes a set of rails 235 on opposing sides, configured to slidably receive prongs 240 of the base 225 of the actuator housing 205. The prongs 240 and the sides of the reservoir housing 210 have corresponding apertures 245 configured to receive fasteners 250. In the illustrated embodiment, the fasteners 250 are shown as screws. However, it should be understood that any fasteners may be employed. Exemplary fasteners include bolts, pins, ties, and other known fasteners. In an alternative embodiment (not shown), the apertures and fasteners may be omitted. Instead, the reservoir housing 210 may be attached to the actuator housing 205 by a press fit, a snap fit, clamps, or other attachment means.

The reservoir housing 210 includes two input lines (not shown) and two output lines 255. The two input lines may be connected to a single input line (not shown) by a y-connector (not shown). Similarly, the two output lines 255 may be connected to a single output line (not shown) by a y-connector (not shown).

The intravenous fluid delivery device 200 may be constructed of various materials. Exemplary materials include polymeric materials and metal materials. Exemplary metal materials include, without limitation, steel, nickel aluminum, copper, iron, and other metals and alloys. Exemplary polymeric materials include, without limitation, EPDM rubber, latex, polypropylene, polyethylene, and blends of the same. In one embodiment, where the intravenous fluid delivery device is configured for field use (i.e., in an ambulance, or at an accident site), the device may be constructed of materials that are lightweight and durable. Of course, such materials may also be suitable for a device configured for clinical use. In one embodiment, the actuator housing 205, the reservoir housing 210, and the internal components are all constructed of substantially the same material. In an alternative embodiment, one or more of these components are constructed of different materials.

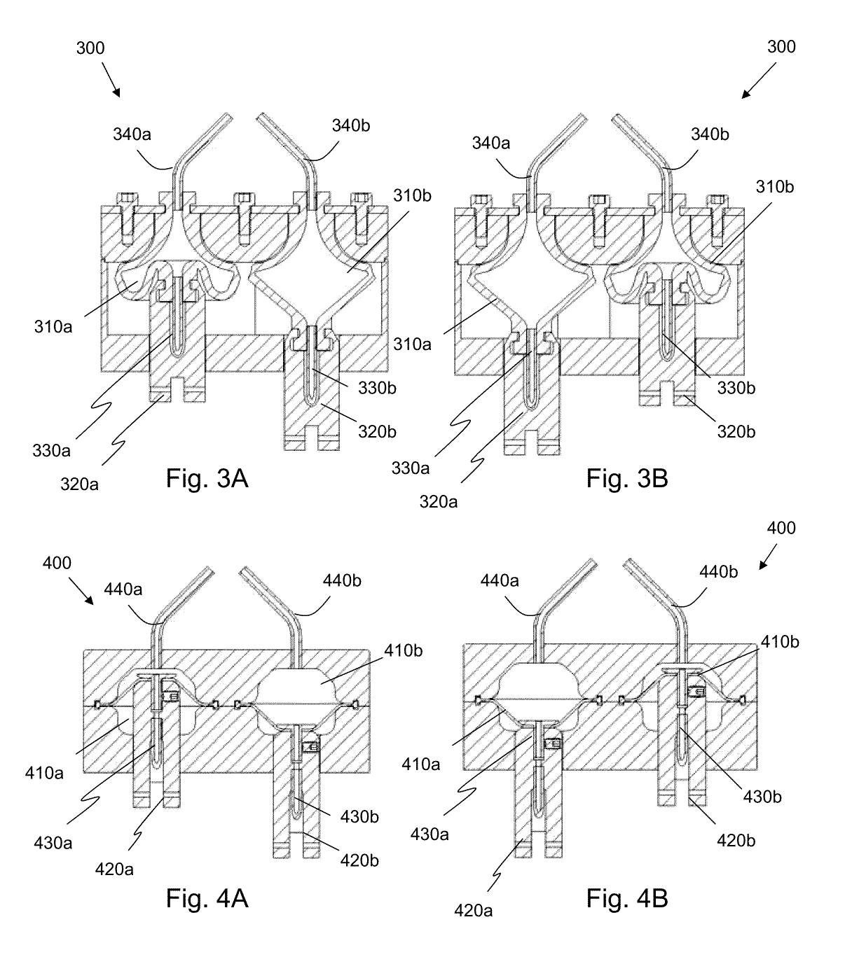

The internal components of two exemplary embodiments of reservoir housings 210 are shown in FIGS. 3A, 3B, 4A, and 4B.

FIGS. 3A and 3B illustrate cross-sections of one embodiment of a reservoir housing 300. The reservoir housing 300 includes two fluid reservoirs defined by a first asymmetric diaphragm pump 310a and a second asymmetric diaphragm pump 310b. The first and second asymmetric diaphragm pumps 310a,b are collapsible bellows or diaphragms that inflate and deflate with fluid. The first asymmetric diaphragm pump 310a is connected to a first piston 320a, a first input line 330a, and a first output line 340b. The second asymmetric diaphragm pump 310b is connected to a second piston 320b, a second input line 330b, and a second output line 340b.

In the illustrated embodiment, the first asymmetric diaphragm pump 310a is out of phase with the second asymmetric diaphragm pump 310b. When the first piston 320a collapses the first asymmetric diaphragm pump 310a, as shown in FIG. 3A, fluid in the first asymmetric diaphragm pump 310a is forced through the first output line 340a. The second piston 320b opens the second asymmetric diaphragm pump 310b concurrently, and fluid flows through the second input line 330b into the second asymmetric diaphragm pump 310b. As the cycle continues, as shown in FIG. 3B, the second piston 320b collapses the second asymmetric diaphragm pump 310b, forcing fluid out of the second diaphragm pump 310b and through the second output line 340b. The first piston 320a opens the first asymmetric diaphragm pump 310a concurrently, and fluid flows through the first input line 330a into the first asymmetric diaphragm pump 310a. Each of the first and second asymmetric diaphragm pumps 310a,b may have check valves (not shown) associated therewith.

In one embodiment, fluid would flow through the asymmetric diaphragm pumps 310a,b and the output lines 340a,b, even when the pumps were not being actuated. In an alternative embodiment, fluid would only flow through the asymmetric diaphragm pumps 310a,b upon actuation. In another alternative embodiment (not shown), the system includes a flow regulation mechanism (i.e., a safety, or an on/off switch) that would allow an operator to prevent fluid from flowing through the output lines 340a,b. Such a flow regulation mechanism may be located on the reservoir housing 300.

In an alternative embodiment (not shown), the first and second asymmetric diaphragm pumps 310a,b may operate in phase. In another alternative embodiment (not shown), the reservoir housing 300 includes a single asymmetric diaphragm pump. In yet another alternative embodiment (not shown), the reservoir housing 300 includes three or more asymmetric diaphragm pumps.

FIGS. 4A and 4B illustrate cross-sections of another embodiment of a reservoir housing 400. The reservoir housing 400 is substantially the same as the reservoir housing 400, except that it includes two fluid reservoirs defined by a first symmetric diaphragm pump 410a and a second symmetric diaphragm pump 410b. The first and second symmetric diaphragm pumps 410a,b are collapsible bellows or diaphragms that inflate and deflate with fluid. The first symmetric diaphragm pump 410a is connected to a first piston 420a, a first input line 430a, and a first output line 440b. The second symmetric diaphragm pump 410b is connected to a second piston 420b, a second input line 430b, and a second output line 440b.

In the illustrated embodiment, the first symmetric diaphragm pump 410a is out of phase with the second symmetric diaphragm pump 410b, and the pumps operate in the same manner as described in FIGS. 4A and 4B. In an alternative embodiment (not shown), the first and second symmetric diaphragm pumps 410a,b may operate in phase. In another alternative embodiment (not shown), the reservoir housing 400 includes a single symmetric diaphragm pump. In yet another alternative embodiment (not shown), the reservoir housing 400 includes three or more symmetric diaphragm pumps.

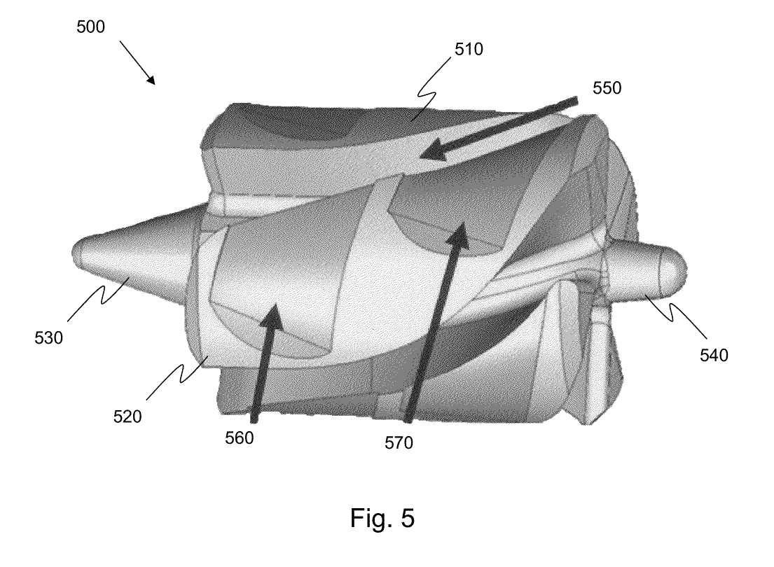

FIG. 5 illustrates a cut away view of an alternative embodiment of a pump 500 for use in an intravenous fluid delivery device. The pump 500 is an axial flow pump having an outer housing 510 and a bladed rotor 520, and may be employed with any embodiment of an intravenous fluid delivery device described herein. The pump 500 may be employed as a single pump, or in combination with one more additional pumps.

In the illustrated embodiment, the outer housing 510 has a first a projection 530 along a first axis and the rotor has a second projection 540. The second projection 540 may be located on the first axis, or it may be located along a second axis different from the first axis. The bladed rotor 520 is disposed in the outer housing 510 in a manner providing clearance between an outer surface of the bladed rotor 520 and an inner surface of the outer housing 510. This clearance defines one or more flow channels 550 for a fluid.

The bladed rotor 520 further includes at least one hydrodynamic bearing. In the illustrated embodiment, the rotor includes a first hydrodynamic bearing 560 and a second hydrodynamic bearing 570. The first and second hydrodynamic bearings 560, 570 are larger and wider than the area between blades where fluid flows. In an alternative embodiment (not shown), the first and second hydrodynamic bearings 560, 570 are narrower than the area between blades where fluid flows.

The bladed rotor 520 is configured to rotate within the outer housing 510, thereby facilitating a flow of fluid. The bladed rotor 520 may be rotated by activation of an electric motor or with the use of magnets or electronics.

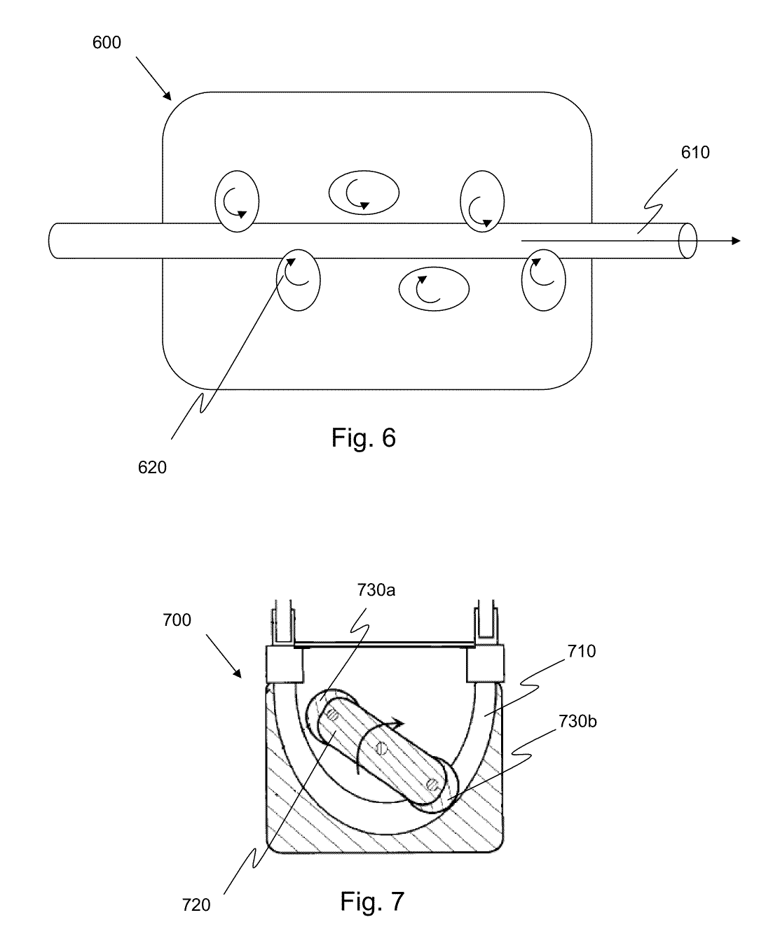

FIG. 6 illustrates a cross section of a fluid line housing 600 for an intravenous fluid delivery device. In the illustrated embodiment, the fluid line housing 600 that can be used with an actuator housing instead of a reservoir housing. However, it should be understood that the fluid line housing may be incorporated in a single housing that includes both an actuator and a fluid line.

The fluid line housing 600 includes a fluid line 610. In the illustrated embodiment, the fluid line 610 is a single line that is partially disposed within the housing 600, but extends beyond the boundaries of the housing. The fluid line 610 has a first end operably connected to a fluid source (not shown) and a second end operably connected to an intravenous needle (not shown). The fluid line 610 may be disposed in a slot in the fluid line housing 600 such that the fluid line 610 may be inserted and removed from the fluid line housing 600 without any of the fluid coming into contact with the fluid line housing 600. In such an embodiment, the fluid line housing 600 may be reused with multiple fluid lines for multiple patients without contamination. In an alternative embodiment (not shown), the fluid line is disposed inside the fluid line housing and has a first end configured to be connected to an input line and a second end configured to be connected to an output line.

The fluid line housing 600 further includes a plurality of rollers 620. In the illustrated embodiment, the fluid line housing 600 includes six rollers 620. However, it should be understood that any number of rollers may be employed. Each of the rollers 620 is operably connected to a motor, such as through a series of gears. Each roller 620 is slightly elongated, such that as each roller turns, it comes into and out of engagement with the fluid line 610. The part of fluid line 610 under compression closes (or occludes) thus forcing the fluid to move through the fluid line 610. When the roller comes out of engagement with the fluid line 610, the fluid line opens to its natural state and fluid flow is induced to that section of the fluid line 610. Such a process may be referred to as “peristalsis” and the rollers 620 may therefore be referred to as “peristaltic rollers.”

FIG. 7 illustrates a cross section of an alternative embodiment of a fluid line housing 700 for an intravenous fluid delivery device. It should be understood that the details of the illustrated fluid line housing may also be incorporated in a single housing that includes both an actuator and a fluid line.

The fluid line housing 700 includes a fluid line 710. In the illustrated embodiment, the fluid line 710 is disposed inside the fluid line housing and has a first end configured to be connected to an input line and a second end configured to be connected to an output line. In an alternative embodiment (not shown), the fluid line is a single line that is partially disposed within the housing, but extends beyond the boundaries of the housing. In such an embodiment, the fluid line may be disposed in a slot in the fluid line housing.

The fluid line 710 has an arcuate shape, such that the first end and second end of the fluid line 710 are both disposed on the same side of the fluid line housing 700.

The fluid line housing 700 further includes a rotary device 720. Although the rotary device 720 is elongated and non-circular, it may still be referred to as a “roller.” The rotary device 720 is operably connected to a motor, such as through a series of gears. The rotary device 720 is elongated, such that as each roller turns, it comes into and out of engagement with the fluid line 710. Due to the arcuate shape of the fluid line 710, the rotary device 720 may come into engagement with two sections of the fluid line 710 at the same time. In alternative embodiments (not shown), the rotary device may have three or more ends that come into engagement with the fluid line.

In the illustrated embodiment, the first end of the rotary device 720 has a first roller 730a rotatably connected thereto and the second end of the rotary device 720 has a second roller 730b rotatably connected thereto. In an alternative embodiment (not shown), the rotary device does not include rollers and its ends directly engage the fluid line.

Rotation of the rotary device 720 causes a similar peristaltic process as described above with respect to FIG. 6.

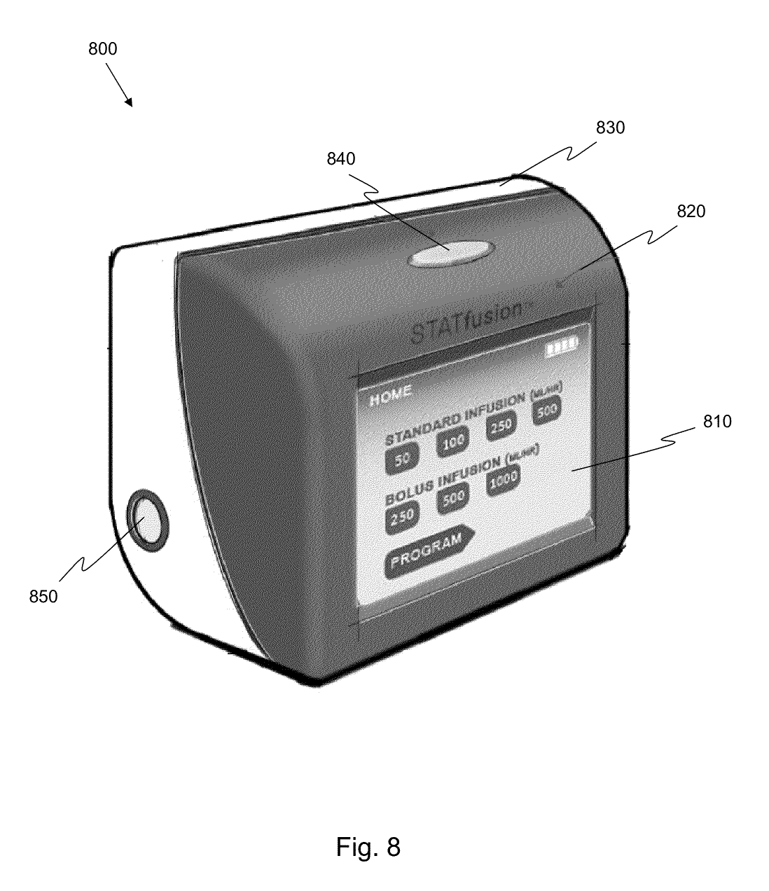

FIG. 8 is a front perspective view of one embodiment of an intravenous fluid delivery device 800 having a user interface 810. The intravenous fluid delivery device 800 includes an actuator housing 820 and a reservoir housing 830, that may be substantially similar to the actuator housing 205 and reservoir housing 210 described above with reference to FIG. 2. The actuator housing 820 includes a motor (not shown) and a controller operably connected to the motor (not shown). The controller is configured to actuate the motor and to control the speed of the motor.

The user interface 810 is disposed on the actuator housing 820, and is operably connected to the controller. In the illustrated embodiment, the user interface 810 is a touch screen. The user interface also includes a button 840. In one embodiment, the button 840 is a “home” key that will cause the touch screen to display a “home” screen. In an alternative embodiment, the button 840 is an “enter” key or “OK” key, that can be used to confirm a user selection. In an alternative embodiment (not shown), the user interface is an LCD display and a plurality of buttons. In another alternative embodiment (not shown), the user interface includes any combination of buttons, keys, dials, LED indicators, and other inputs and displays.

The reservoir housing 830 includes at least one fluid input line, having a first end 850 and a second end (not shown). The first end 850 is configured to be operably connected to a fluid source that is external to the actuator housing and external to the reservoir housing. The reservoir housing 830 further includes at least one fluid reservoir (not shown) operably connected to the second end of the at least one fluid input line, and at least one fluid output line operably connected to the at least one fluid reservoir. The fluid lines and fluid reservoir may be substantially similar to those described above with reference to FIGS. 3A and 3B.

The reservoir housing 830 further includes at least one pump configured to facilitate a first flow of fluid from the input line to the fluid reservoir and a second flow of fluid from the fluid reservoir to the output line. The at least one pump is operably connected to the motor. The pump may be substantially similar to those pumps described above with reference to FIGS. 3-7.

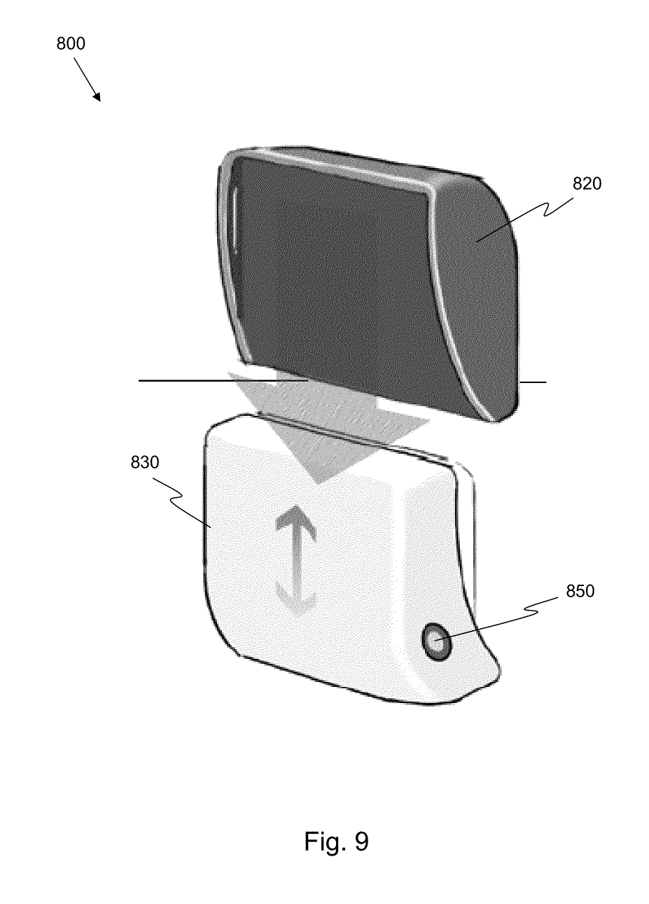

FIG. 9 is an exploded rear perspective view of the intravenous fluid delivery device 800 of FIG. 8. As can be seen from this view, the actuator housing 820 is configured to slidably engage and slidably disengage the reservoir housing 830. In one embodiment, the actuator housing 820 is configured to be reusable and the reservoir housing 830 is configured to be disposable. As explained above with reference to FIG. 2, the reservoir housing 830 may be replaced after each use for sterility or safety reasons, or to comply with FDA standards, hospital standards, or other standards. In such an embodiment, the reservoir housing 830 may be kept in sterile packaging prior to use. Additionally, the reservoir housing 830 may be filled with fluid prior to packaging, such that no priming is required when a new reservoir housing 830 is attached to the actuator housing 820. After being used, the reservoir housing 830 may be disposed, recycled, or refurbished. It should be understood that refurbishing the reservoir housing 830 may include sterilizing the reservoir housing 830.

In one embodiment, a power source (not shown) is disposed in the reservoir housing 830. Although the power source is disposed in the reservoir housing 830, it is operably connected to the motor and the user interface 810 in the actuator housing 820. Where the reservoir housing 830 is a disposable or single-use component, locating the power source in the reservoir housing 830 may obviate the need for a user to replace or recharge the power source. Where the reservoir housing 830 is refurbished, the refurbishing process may include replacing or recharging the power source. However, in an alternative embodiment, the power source is disposed in the actuator housing. Exemplary power sources include batteries and solar cells.

The fluid delivery device 800 further includes one or more sensors configured to detect presence of air in one of the fluid input line, the fluid reservoir, and the fluid output line. In one embodiment, at least one sensor is disposed on the actuator housing 810. In an alternative embodiment, at least one sensor is disposed on the reservoir housing 820.

In one embodiment, the sensors are operably connected to the controller. The controller is configured to stop the motor upon receiving a signal from a sensor indicating the presence of air in one of the fluid input line, the fluid reservoir, and the fluid output line. Additionally, the user interface 810 is configured to display a notification upon receiving a signal from a sensor indicating the presence of air in one of the fluid input line, the fluid reservoir, and the fluid output line. Exemplary sensors include optical sensors and tactile sensors.

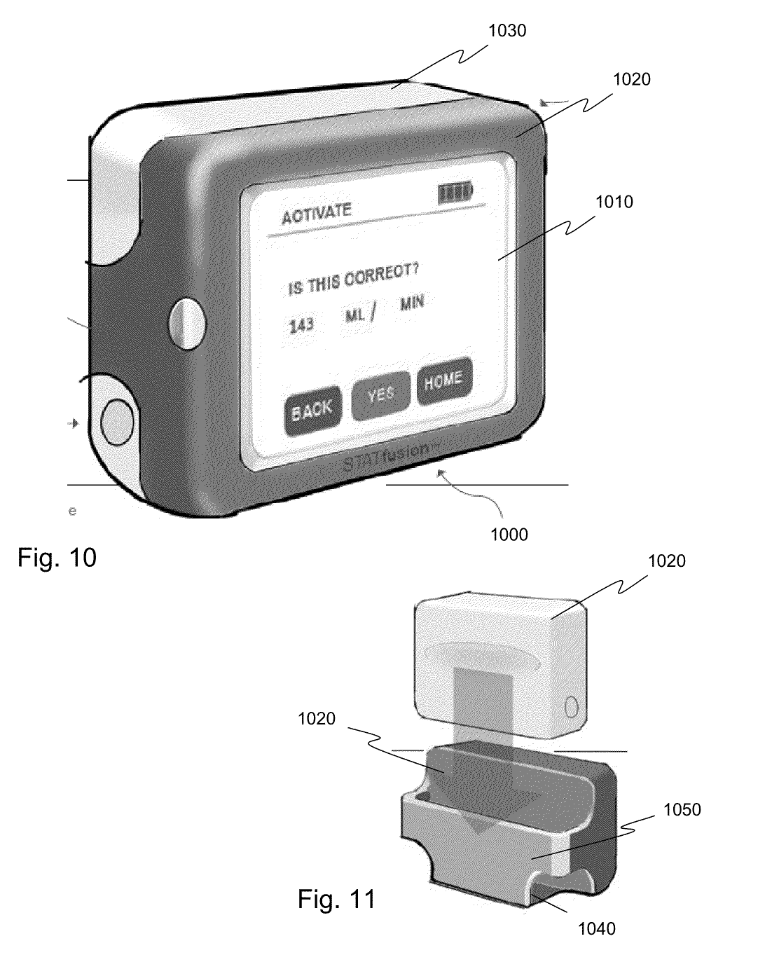

FIG. 10 is a front perspective view of an alternative embodiment of an intravenous fluid delivery device 1000 having a user interface 1010. FIG. 11 is an exploded rear perspective view of the alternative intravenous fluid delivery device 1000. The intravenous fluid delivery device 1000 is substantially the same as the intravenous fluid delivery device 800 described above with reference to FIGS. 8 and 9, except for the differences described herein.

The intravenous fluid delivery 1000 includes an actuator housing 1020 and a reservoir housing 1030 that slidably engages and slidably disengages the actuator housing 1020. The actuator housing 1020 includes a ledge 1040 and a rear support 1050, which together are configured to receive and retain the reservoir housing 1030.

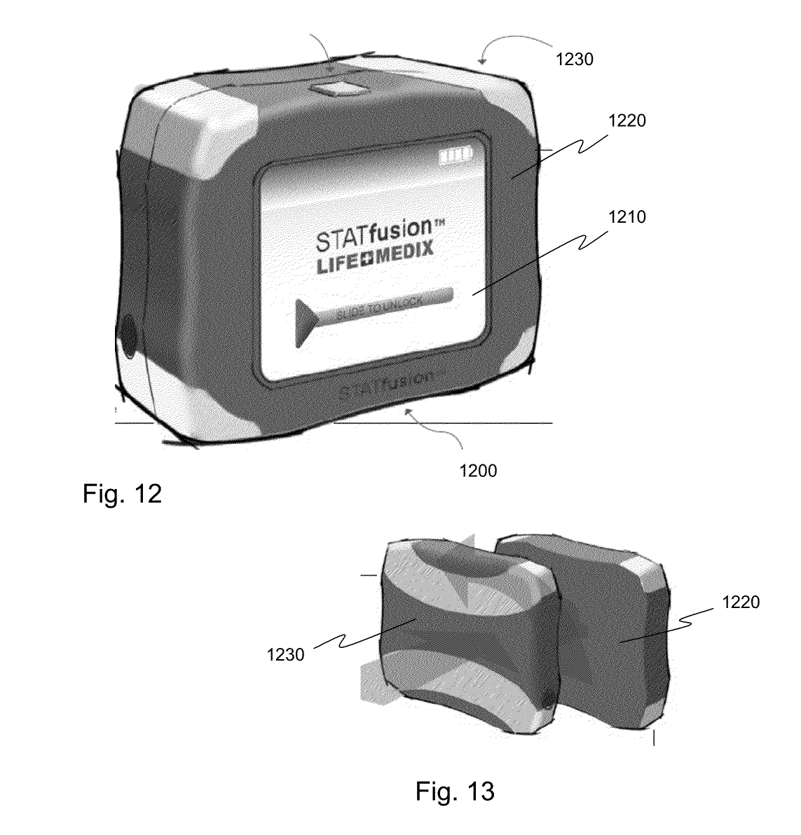

FIG. 12 is front perspective view of another alternative embodiment of an intravenous fluid delivery device 1200 having a user interface 1210. FIG. 13 is an exploded rear perspective view of the alternative intravenous fluid delivery device 1200. The intravenous fluid delivery device 1200 is substantially the same as the intravenous fluid delivery devices 800 and 1000 described above with reference to FIGS. 8-11 except for the differences described herein.

The intravenous fluid delivery 1200 includes an actuator housing 1220 and a reservoir housing 1230 that rotatably engages and rotatably disengages the actuator housing 1220.

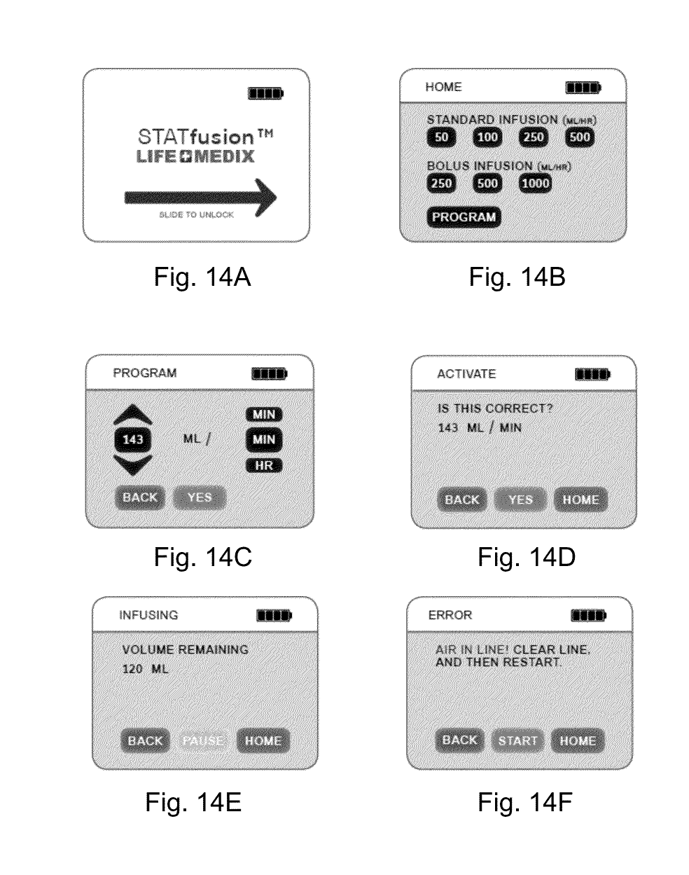

FIGS. 14A-F are schematic drawings of exemplary displays of a user interface. FIG. 14A illustrates one embodiment of a lock screen for a touch screen. A lock screen is a screen that only accepts a predetermined user input to prevent accidental actuation of a device. In the illustrated embodiment, a user unlocks the lock screen by sliding a figure from left to right across the arrow. In an alternative embodiment (not shown), the user unlocks the lock screen by moving one or more fingers along a predetermined path. In an alternative embodiment (not shown), the user unlocks the lock screen by typing a pin or password. In another alternative embodiment, the user unlocks the lock screen by pressing the screen for a predetermined length of time.

FIG. 14B illustrates one embodiment of a home screen. A home screen is a first screen displayed to a user after unlocking a lock screen, and a screen that the user returns to by pressing a home key. In the illustrated embodiment, the home screen displays a plurality of choices of fluid flow rates. The flow rates a displayed in rates of milliliters per hour, and are grouped according to standard infusion rates and bolus infusion rates. However, it should be understood that the fluid flow rates may be displayed in other units, and may be grouped in any manner desired. Upon selection of a fluid flow rate by a user, the controller actuates the motor at a speed corresponding to the selected fluid flow rate.

The home screen also displays a program button, that allows the user to select a fluid flow rate other than those displayed on the home screen. FIG. 14C illustrates one embodiment of a program screen. In the illustrated embodiment, a user selects a fluid flow rate by pressing arrow buttons to increase or decrease the flow rate. Additionally, the user may select a flow rate in terms of milliliters per minute or milliliters per hour. In alternative embodiments (not shown), the user may select other units for flow rates. In another alternative embodiment (not shown), the user interface displays a keypad, and the user may select digits for a fluid flow rate.

After the desired fluid flow rate has been entered in the program screen, the user hits the “YES” icon and an activation screen is displayed. FIG. 14D illustrates an exemplary activation screen. The activation screen displays a confirmation message to ensure that the entered flow rate is the desired flow rate. The user may confirm the flow rate by pressing the “YES” icon, or the user may press the “BACK” icon to return to the previous screen, or the “HOME” icon to return to the home screen.

While the fluid delivery device is delivering fluids to a subject, the user interface may display an infusion screen, such as the infusion screen shown in FIG. 14E. The infusion screen indicates that fluid is being delivered, and may display an amount of fluid remaining in the fluid source. In the illustrated embodiment, the infusion screen displays a number of milliliters remaining In an alternative embodiment (not shown), the infusion screen may display a progress bar, or a time icon such as a clock or an hourglass. The infusion screen also displays a “PAUSE” icon that allows the user to pause fluid delivery, and a “BACK” and “HOME” icon to allow a user to view other display screens.

FIG. 14F illustrates an exemplary error screen. In the illustrated embodiment, the error screen displays a message that air was detected in the fluid path. In other embodiments (not shown), error screens may be displayed for other purposes, including, without limitation, to indicate a low battery status, a resistance or stoppage on the motor, a fault with the pump, a kinked fluid line, or a change in pressure.

As one of ordinary skill would understand, additional screens may be displayed by the touch screen for other purposes.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

What is claimed is:

1. A portable intravenous fluid delivery system comprising:
an actuator housing including:
a motor,
a controller operably connected to the motor, the controller being configured to actuate the motor and to control a speed of the motor, and
a user interface operably connected to the controller; and
a reservoir housing removably attached to the actuator housing, wherein the reservoir housing includes:
at least one fluid input line, having a first end and a second end, the first end configured to be operably connected to a fluid source that is external to the actuator housing and external to the reservoir housing,
at least one fluid reservoir operably connected to the second end of the at least one fluid input line,
at least one fluid output line operably connected to the at least one fluid reservoir,
at least one pump configured to facilitate a first flow of fluid from the at least one fluid input line to the at least one fluid reservoir and a second flow of fluid from the at least one fluid reservoir to the at least one fluid output line, the at least one pump being operably connected to the motor, and
a power source operably connected to the motor in the actuator housing.

2. The system of claim 1, wherein the at least one fluid input line includes a first fluid input line and a second fluid input line, the at least one fluid reservoir includes a first fluid reservoir and a second fluid reservoir, the at least one fluid output line includes a first fluid output line and a second fluid output line, and the at least one pump includes a first pump operably connected to the first fluid reservoir and a second pump operably connected to the second fluid reservoir.

3. The system of claim 2, wherein the first pump is configured to operate out of phase with the second pump.

4. The system of claim 1, wherein the user interface is a touch screen.

5. The system of claim 4, wherein the user interface displays a plurality of fluid flow rates, and wherein upon selection of a fluid flow rate, the controller actuates the motor at a speed corresponding to the selected fluid flow rate.

6. The system of claim 1, further comprising a sensor configured to detect presence of air in one of the at least one fluid input line, the at least one fluid reservoir, and the at least one fluid output line.

7. The system of claim 6, wherein the sensor is operably connected to the controller, and wherein the controller is configured to stop the motor upon receiving a signal from the sensor indicating the presence of air in one of the at least one fluid input line, the at least one fluid reservoir, and the at least one fluid output line.

8. The system of claim 6, wherein the sensor is operably connected to the user interface, and wherein the user interface is configured to display a notification upon receiving a signal from the sensor indicating the presence of air in one of the at least one fluid input line, the at least one fluid reservoir, and the at least one fluid output line.

9. The system of claim 6, wherein the sensor is disposed in the actuator housing.

10. A fluid delivery device comprising:
a first housing;
a second housing removably connected to the first housing;
an input line operatively connected to a fluid source that is external to each of the first housing and the second housing;
a fluid reservoir operably connected to the input line;
an output line operably connected to the fluid reservoir;
a pump configured to facilitate a flow of fluid from the input line to the fluid reservoir and from the fluid reservoir to the output line;
a motor operably connected to the pump;
a user interface; and
a power source operably connected to the motor and to the user interface.

11. The fluid delivery device of claim 10, wherein the input line, the fluid reservoir, the output line, the pump, and the power source are disposed in the first housing.

12. The fluid delivery device of claim 11, wherein the motor is disposed in the second housing and the user interface is disposed on the second housing.

13. The fluid delivery device of claim 10, wherein the first housing is configured to slidably engage and slidably disengage the second housing.

14. The fluid delivery device of claim 10, wherein the first housing is configured to rotatably engage and rotatably disengage the second housing.

15. A portable intravenous fluid delivery kit comprising:
a first housing including:
an electric motor, and
a user interface;
a second housing including:
a fluid input;
a fluid reservoir operably connected to the fluid input,
a fluid output operably connected to the fluid reservoir,
a pump configured to be connected to the electric motor, and
a power source configured to be connected to the electric motor and the user interface;
a fluid bag external to the first housing and second housing; and
a fluid line having a first end connected to the fluid bag and a second end connected to the fluid input of the second housing.

16. The portable intravenous fluid delivery kit of claim 15, further comprising a second fluid line having a first end connected to the fluid output of the second housing.

17. The portable intravenous fluid delivery kit of claim 15, further comprising a sensor configured to monitor one of the fluid input, the fluid reservoir, and the fluid output.

18. The portable intravenous fluid delivery kit of claim 17, wherein the sensor is an optical sensor.

19. The portable intravenous fluid delivery kit of claim 15, wherein the user interface is a touch screen having a lock screen and a home screen.

20. The portable intravenous fluid delivery kit of claim 19, wherein the user interface is configured to display a volume of fluid remaining in the fluid bag.
